(19) United States
(12) Patent Application Publication
(10) Pub. No.: US 2016/0041172 A1
(43) Pub. Date: Feb. 11, 2016

(54) COMPOSITION FOR DETECTION OR DIAGNOSIS OF DISEASES CONTAINING TRANSCRIPTION ACTIVATOR-LIKE EFFECTOR

(71) Applicant: KOREA INSTITUTE OF SCIENCE AND TECHNOLOGY, Seoul (KR)

(72) Inventors: Kwan Hyi LEE, Seoul (KR); Hyun Kwang Seok, Seoul (KR); Seok Lee, Seoul (KR); Gunhyuk Jang, Seoul (KR); Minhong Jeun, Yangju-si (KR)

(73) Assignee: KOREA INSTITUTE OF SCIENCE AND TECHNOLOGY, Seoul (KR)

(21) Appl. No.: 14/486,251

(22) Filed: Sep. 15, 2014

(30) Foreign Application Priority Data
Aug. 7, 2014 (KR) 10-2014-0101686

(51) Int. Cl. G01N 33/574; G01N 33/569

(52) U.S. Cl. CPC G01N 33/57434; G01N 33/56983; G01N 33/57438; G01N 33/57419; G01N 33/57411; G01N 2333/11

(57) ABSTRACT

The present disclosure relates to a composition or a kit that can be used for detection or diagnosis of various diseases.

### CROSS-REFERENCE TO RELATED APPLICATION

This application claims foreign priority benefit under 35 U.S.C. §119 of Korean Patent Application No. 10-2014-0101686, filed on Aug. 7, 2014 in the Korean Intellectual Property Office, the contents of which are herein incorporated by reference.

### BACKGROUND

1. Field

The present disclosure relates to a composition or a kit that can be used for detection or diagnosis of various diseases.

2. Description of the Related Art

Researches are under way on techniques for early detection of diseases caused by pathogenic bacteria or viruses and their genetic effects. Examples include detection of specific proteins, i.e., surface markers or antigens (Biosensors & Bioelectronics, vol. 34, pp. 12-24, Apr. 15 2012; ACS Nano, vol. 7, pp. 4967-4976, Jun. 25 2013), detection of pathogenic bacteria through culturing (Nature Reviews Gastroenterology & Hepatology, vol. 9, pp. 312-322, Mar. 27 2012), detection of DNAs through PCR using tailored primers (Science, vol. 314, pp. 1464-1467, Dec. 1 2006; Nature Nanotechnology, vol. 8, pp. 369-375, May 5 2013) and detection by attaching onto beads or particles (Biosensors & Bioelectronics, vol. 29, pp. 46-52, Nov. 15 2011).

However, the detection methods targeting surface markers or antigens are limited in terms of time and cost if relevant antigens or antibodies are unavailable (The Medical Clinics of North America, vol. 96, pp. 1067-1078, November 2012) and the detection methods based on culturing are also restricted a lot in terms of time and cost (Nature Nanotechnology, vol. 8, pp. 369-375, May 5 2013).

The DNA-based detection methods are useful since they can be applied in most cases irrespective of the subjects. At present, DNAs are detected by preparing dozens of single-stranded oligoprimers and conducting PCR based on their complementary binding (Science, vol. 314, pp. 1464-1467, Dec. 1 2006; Nature Nanotechnology, vol. 8, pp. 369-375, May 5 2013) or by attaching them onto particles (Biosensors & Bioelectronics, vol. 29, pp. 46-52, Nov. 15 2011). These DNA-based detection methods are problematic in that detection is possible only when the target DNA is single-stranded or manipulated to be single-stranded (Nature Nanotechnology, vol. 8, pp. 369-375, May 5 2013). In addition, since the specific bonding between complementary bases occurs at moderately high temperatures (Nature Nanotechnology, vol. 8, pp. 369-375, May 5 2013), an unwanted result may be obtained due to non-specific binding if temperature is not sufficiently high or if the sample binding is not in an optimized state (Nucleic Acids Research, vol. 40, pp. W205-W208, July 2012)

### REFERENCES OF THE RELATED ART

### Non-patent Document

Nature Nanotechnology, vol. 8, pp. 369-375, May 5 2013.

### SUMMARY

The present disclosure is directed to providing a composition or a kit capable of detecting and diagnosing various diseases easily and conveniently.

In an aspect, the present disclosure provides a composition for detection or diagnosis of a disease, containing a dTALE (designed transcription activator-like effector) which binds to a disease-causing gene.

In another aspect, the present disclosure provides a kit for detection or diagnosis of a disease, containing a dTALE (designed transcription activator-like effector) which binds to a disease-causing gene.

The present disclosure is advantageous in that the presence of a specific disease in an individual can be detected conveniently using only the TALE (transcription activator-like effector) of the TALEN (transcription activator-like effector nuclease), which have been used only to cleave a specific base sequence in a target gene or to introduce a new gene.

The composition or kit of the present disclosure is advantageous in that the double-stranded DNA can also be detected. The PCR method currently used for detection of a target DNA requires denaturation of a double-stranded DNA to single strands by applying shock (e.g., heat or acid) since the primer can bind only to a single-stranded DNA. In contrast, the composition or kit of the present disclosure is advantageous in that it can bind to the naturally occurring double-stranded DNA as it is.

In addition, since the composition or kit can accurately and quickly bind to abnormal genes in various diseases, the diseases can be diagnosed or detected easily and conveniently. In particular, by designing a TALE to be capable of binding to an abnormal gene specific for a disease to be detected and loading the same on a commercially available kit, the disease can be self-diagnosed easily and conveniently at low cost.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically shows that a target DNA at a target site can be detected by designing a TALE domain that can bind to the target sequence regardless of its type.

FIGS. 2-4 show exemplary structures of a TALE domain according to the present disclosure.

FIG. 5 schematically shows a procedure of preparing TALE sensors based on the StuI restriction site and the BamHI restriction site in the gene sequence of the T7 bacteriophage (Example 1) and detecting target gene sequences (Test Example 1).

FIG. 6 shows a TALE domain and a tag attached following the TALE C-terminal.

FIG. 7 shows a result of synthesizing a TALE protein by introducing a TALE-expressing vector into E. coli and purifying and electrophoresing the same.

FIG. 8 schematically shows a purified TALE domain protein and a tagging protein site attached on a QD.

FIG. 9 schematically shows isolation of a site binding to a TALE sensor by cleaving the entire 10-3b T7 bacteriophage sequence.

FIG. 10 shows a result of identifying the cleaved 10-3b T7 bacteriophage sequence by electrophoresis.

FIG. 11 shows that a target DNA site can be detected by confirming binding between the purified TALE domain protein and the tagging protein site attached on the QD as shown in FIG. 8 and the 10-3b T7 bacteriophage sequence cleaved as shown in FIG. 9.

FIG. 12 shows a result of detecting T7 fragments using a TALE-QD sensor prepared according to the present disclosure.

### DETAILED DESCRIPTION

As used herein, the term “disease” includes any disease wherein the specific gene sequence which causes the disease is known without limitation.

As used herein, the term “disease-causing gene” refers to a gene sequence known to cause or aggravate the corresponding disease.

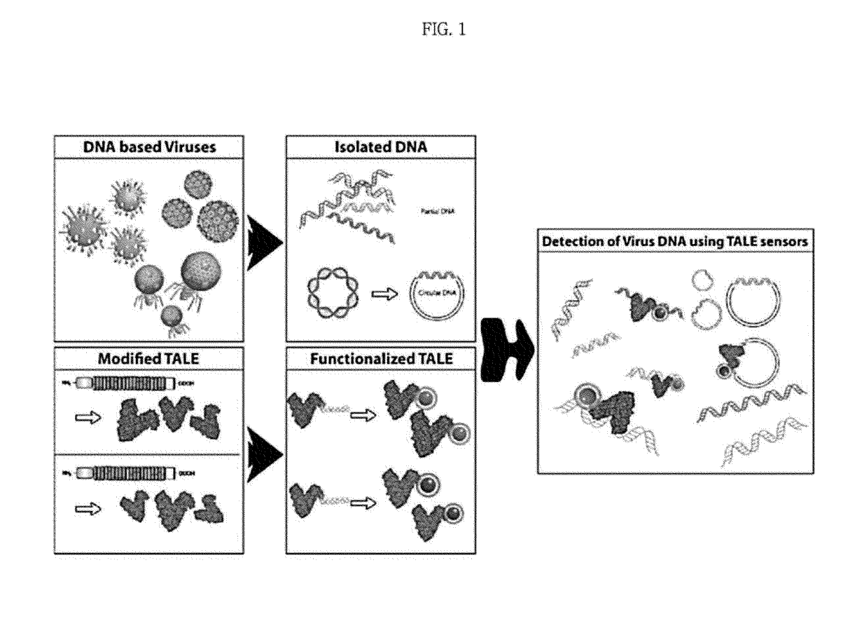

As used herein, the term “gene” sequence includes any sequence including one or more DNA strand. Specifically, a DNA-RNA hybrid complex sequence may be included as in AIDS. The DNA sequence includes both single-stranded and double-stranded sequences as well as circular DNA or free DNA. They are schematically shown in FIG. 1.

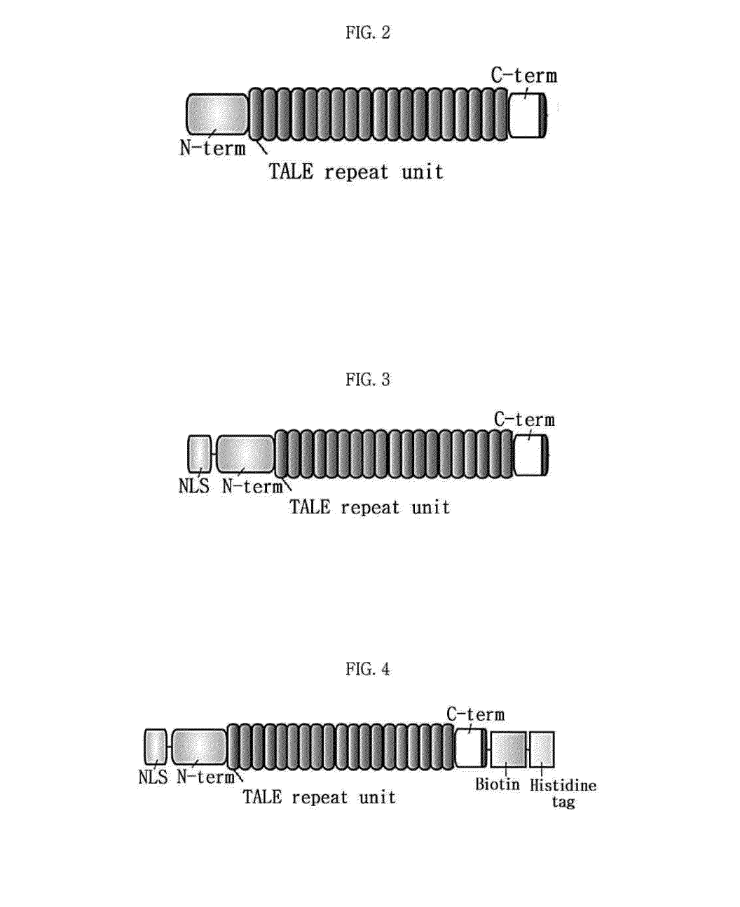

As used herein, the term “TALE (transcription activator-like effector)” refers to a protein secreted by pathogenic Xanthomonas bacteria when they infect various plant cells. The protein can bind promoter sequences in the host plant and activate the expression of plant genes that aid bacterial infection or inhibit the expression of plant genes that interfere with the infection (Current Opinion in Plant Biology, vol. 13, pp. 394-401, August 2010; Science, vol. 326, pp. 1501, Dec. 11 2009). Specifically, in the present disclosure, a “TALE domain” may include two or more TALE repeat units, more specifically 2-30 TALE repeat units. If the TALE domain includes less than 2 TALE repeat units, it is difficult to be designed into a structure for detecting a target gene. And, if the TALE domain includes more than 30 TALE repeat units, the TALE domain becomes too large in size for detection using, for example, a nanostructure. In this regard, the TALE domain may include 3 or more, 4 or more, 5 or more, 6 or more, 7 or more, 8 or more, 9 or more or 10 or more TALE repeat units, or may include 29 or less, 28 or less, 27 or less, 26 or less, 25 or less, 24 or less, 23 or less, 22 or less, 21 or less or 20 or less TALE repeat units. For example, a TALE domain according to the present disclosure may have a structure as shown in FIG. 2.

In the present disclosure, in order to diagnose a specific disease by transporting the TALE domain, for example, into the nucleus, an NLS (nuclear localization signal) domain may be further bound at the N-terminal. It may be unnecessary if the subject of detection is blood or urine. FIG. 3 shows an exemplary structure of a TALE domain for transporting into the nucleus.

Also, the TALE domain of the present disclosure may further contain a domain widely known in the art for its detection. An example is shown in FIG. 4.

In the present disclosure, the TALE repeat unit consists of 34 amino acids and may specifically bind to a DNA depending on the 12th and 13th amino acid sequences (Science, vol. 326, pp. 1509-1512, Dec. 11 2009; Science, vol. 335, pp. 720-723, Feb. 10 2012). For example, TALE repeat units having the amino acid sequences shown in Table 1 can specifically bind to DNA bases.

TABLE 1

| Nucleotide | | |
|---|---|---|
| bound | Amino acid sequence | SEQ ID NO |
| A | LTPEQVVAIAS NI | 1 |
| | GGKQALETVQRLLPVLCQAHG | |
| T, mC | LTPEQVVAIAS NG | 2 |
| | GGKQALETVQRLLPVLCQAHG | |
| G | LTPEQVVAIAS NN | 3 |
| | GGKQALETVQRLLPVLCQAHG | |
| C | LTPEQVVAIAS HD | 4 |
| | GGKQALETVQRLLPVLCQAHG | |

In the present disclosure, the term “dTALE (transcription activator-like effector)” refers to a TALE designed to be capable of specifically binding to a predetermined DNA sequence. Specifically, the dTALE of the present disclosure may be designed to be capable of specifically binding to a disease-causing gene known in the art. Any designing method known in the art can be employed without limitation. Specifically, the method disclosed in PLoS One, vol. 6, issue 5, e19722. May 19, 2011 may be used. More specifically, the dTALE of the present disclosure may be obtained by any method known in the art, for example, by synthesizing amino acids based on amino acid sequence information and connecting them or by designing a plasmid vector having DNA sequence information so that it can transcript an amino acid sequence and expressing it using the target DNA sequence information (Michael R. Green & Joseph Sambrook. Molecular Cloning, 4th Edition. 2012; PNAS, vol. 96 no. 18, pp. 10068-10073, Aug. 31 1999; Chem Soc Rev. Vol. 41, pp. 7001-15, Nov. 7 2012.). Also, it can be obtained using a commercially available TALEN kit. More specifically, the dTALE of the present disclosure may be obtained by preparing a TALE that can bind to a target gene on a commercially available TALEN kit and expressing proteins after separating it from the TALEN kit. The preparation and separation of the TALE and expression into proteins can be conducted according to any method known in the art.

In an aspect, the present disclosure provides a composition for detection or diagnosis of a disease, containing a dTALE (designed transcription activator-like effector) domain which binds to a disease-causing gene.

In another aspect, the present disclosure provides a kit for detection or diagnosis of a disease, containing a dTALE (designed transcription activator-like effector) domain which binds to a disease-causing gene.

According to the present disclosure, the presence of a disease-causing gene can be detected conveniently owing to the specific binding ability of the TALE repeat unit.

The disease-causing gene refers to a gene which exhibits difference in a normal person and in a patient and may be any one known in the art. Specifically, in the present disclosure, the disease-causing gene may be HPV16 E6 genes (specifically HPV16 E6-1 gene, HPV16 E6-2 gene and/or HPV16 E6-3 gene) among the HPV (human papillomavirus) genes known to cause cervical cancer, hypermethylation sites (ICMT-HES3 gene and/or TBR1 gene) known to cause prostate cancer, BNC1 methylation sites (BNC1-1 gene and/or BNC1-2 gene) known to cause pancreatic cancer, APC gene (NM_{—}000038.5) known to cause colorectal cancer, H7N9 or H5N1 virus gene (more specifically NA (neuraminidase) gene) of avian influenza virus, VP1 gene of coxsackie A5 virus (coxsackievirus A5 isolate PUMCH5454Jun07 VP1 gene) causing hand, foot and mouth disease or gag gene of HIV (HIV-1 isolate P6B_acute_A1 gag protein (gag) gene) known to cause AIDS. In the examples of the present disclosure, dTALE (designed transcription activator-like effector) domain including each of the 20 TALE repeat units having the amino acid sequences shown in Table 2 were prepared to detect the specific sequences known as disease-causing sequences of the genes described above.

The composition or kit according to the present disclosure may contain 2-30 TALE repeat units. If the composition or kit contains less than 2 TALE repeat units, it is difficult to be designed into a structure for detecting a target gene. And, if it contains more than 30 TALE repeat units, the resulting complex becomes too large in size for detection using, for example, a nanostructure. In this regard, the composition or kit may contain 3 or more, 4 or more, 5 or more, 6 or more, 7 or more, 8 or more, 9 or more or 10 or more TALE repeat units, or may contain 29 or less, 28 or less, 27 or less, 26 or less, 25 or less, 24 or less, 23 or less, 22 or less, 21 or less or 20 or less TALE repeat units.

The composition or kit according to the present disclosure may further contain a tag. The dTALE domain unit and the tag may be present in the composition or kit in the form of a complex, and the tag and the dTALE domain unit may bind at a site different from the binding site of the dTALE domain unit with the gene. In the present disclosure, the tag is used to facilitate the detection of the dTALE domain bound to the target DNA and may be any one known in the art which is capable of binding to the amino acid of the dTALE domain without limitation. For example, the tag may be an HIS tag, a CYS tag, a GST tag or a biotin binding tag, although not being limited thereto. Specifically, the biotin binding tag may be avidin, streptavidin, or a biotin binding peptide having an amino acid sequence LAAIPGAGLIGTH.

The composition or kit according to the present disclosure may further contain a detectable labeling agent and the detectable labeling agent may bind to the tag in the complex. The composition or kit according to the present disclosure is advantageous in that the presence or absence of a target DNA can be detected quickly and conveniently since the detectable labeling agent can easily detect the complex of the dTALE domain bound to the target DNA and the tag by binding thereto.

In the present disclosure, the detectable labeling agent may be any one known in the art that can bind to the tag. Specifically, a quantum dot, a magnetic bead nanoparticle, a gold nanoparticle, a fluorescent dye, a fluorescent protein, a nanophosphor or a silicon nanoparticle may be used, although not being limited thereto.

The quantum dot may be one whose surface is made hydrophilic using materials including an amphiphilic material. For example, the amphiphilic material may be one or more selected from a group consisting of MHPC, DPPE-PEG 2000, Ni-NTA and a mixture thereof. The gold nanoparticle refers to a gold particle having a nanometer-sized diameter and is not particularly limited in shape or size. Anyone having shape and size commonly used in the art may be used. For example, the gold nanoparticle may be spherical and have an average particle size of about 2-15 nm. The size of the gold nanoparticle may be defined adequately depending on the shape of the nanoparticle. For example, if the gold nanoparticle is spherical, its diameter is defined as the size. And, if the gold nanoparticle is not spherical, the dimension of the longest axis may be defined as the size. The fluorescent material refers to a substance which allows light-based detection of the target gene. For example, it may be one or more selected from a group consisting of cyanine, rhodamine, Alexa, fluorescein isothiocyanate (FITC), 5-carboxyfluorescein (FAM), Texas Red and fluorescein, although not being limited thereto.

The disease may be cancer, avian influenza, hand, foot and mouth disease or AIDS, and the cancer may be pancreatic cancer, colorectal cancer, cervical cancer or prostate cancer. However, in the present disclosure, the disease is not limited to the above-described diseases. In the present disclosure, the disease includes any disease wherein a gene which exhibits difference in a normal person and in a patient is known.

In the composition or kit according to the present disclosure, the disease-causing gene may contain one or more DNA strand. Specifically, the gene may be one or more selected from a group consisting of a single-stranded DNA, a double-stranded DNA, a circular DNA, a free DNA and a DNA-RNA hybrid.

In another aspect, the present disclosure provides a method for detection or diagnosis of a disease, including treating a sample obtained from an individual with the composition or kit.

As used herein, the term “individual” includes both an individual suspected to have a disease and a normal individual. A disease may be detected or diagnosed by treating a sample obtained from an individual suspected to have the disease individual with the composition or kit of the present disclosure. The same is applied to a normal individual.

As used herein, the term “treating” includes treating a sample obtained from an individual, specifically a sample derived from an individual and isolated from the individual, with the composition or kit.

As used herein, the term “sample” is not limited as long as it is derived from an individual and contains DNA information of the individual. Specifically, it may be blood or urine but is not limited thereto.

In the method for detection or diagnosis of a disease according to the present disclosure, the gene may contain one or more DNA strand.

In the method for detection or diagnosis of a disease according to the present disclosure, the gene may be one or more selected from a group consisting of a single-stranded DNA, a double-stranded DNA, a circular DNA, a free DNA and a DNA-RNA hybrid.

In the method for detection or diagnosis of a disease according to the present disclosure, the disease may be cancer, avian influenza, hand, foot and mouth disease or AIDS, and the cancer may be pancreatic cancer, colorectal cancer, cervical cancer or prostate cancer.

Hereinafter, the present disclosure will be described in detail through examples and test examples. However, the following examples and test examples are for illustrative purposes only and it will be apparent to those of ordinary skill in the art that the scope of the present disclosure is not limited by the examples and test examples.

### Example 1

### Preparation of TALE Domain (dTALE) for Detection of Sequence in T7 Bacteriophage

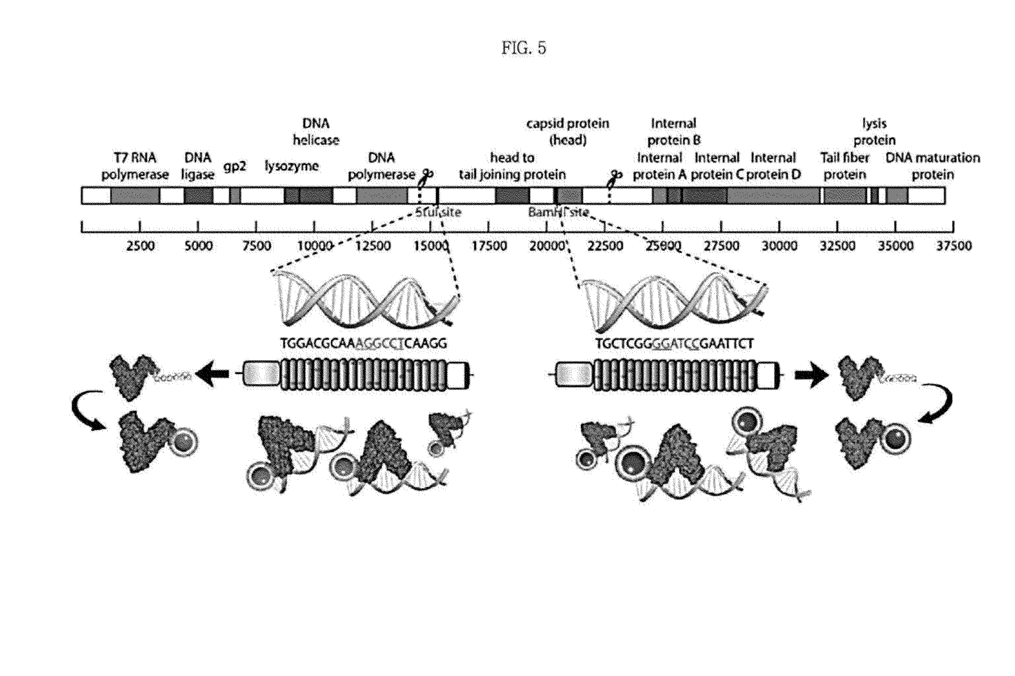

FIG. 5 schematically shows a procedure of preparing TALE sensors based on the StuI restriction site and the BamHI restriction site in the gene sequence of the T7 bacteriophage (Example 1) and detecting target gene sequences (Test Example 1).

### Example 1-1

### Synthesis of TALEN

TALENs were prepared by targeting the site cleaved by the StuI restriction site (NEB #R0187S) (TGGACGCAAAGGCCTCAAGG) and the site cleaved by the BamHI restriction site (NEB #R3136S) (GCTCGGGGATCCGAATTCT) in the gene sequence of the T7 bacteriophage using EZ-TAL Assembly Kit-CMV-TALEN (System Biosciences #SBI-GE100A-1, USA) (Biological Procedures Online, vol. 15, pp. 3, Jan. 14 2013; PLoS One, vol. 6, issue 5, e19722. May 19, 2011). Specifically, since the TALEN backbone vector contains 1st and 20th units among the 20 units, the synthesis was conducted after diving a total of 18 units, from the 2nd to the 19th TALE repeat units, into 3 tubes. 6 of the 18 units were added to each tube together with the restriction enzyme and ligase. PCR was conducted for each combination of the 6 units to obtain amplified samples (35 cycles of initial denaturation at 9° C. for 2 minutes→denaturation at 95° C. for 20 seconds→annealing at 61° C. for 20 seconds→extension at 72° C. for 30 seconds→extension at 72° C. for 3 minutes). Finally, the 3 groups were treated with the restriction enzyme and ligase and combined to link the 18 units. Then, the 18 units were introduced into the TALEN backbone vector to finally obtain a dTALE domain having the 20 units.

### Example 1-2

### Removal of Nuclease

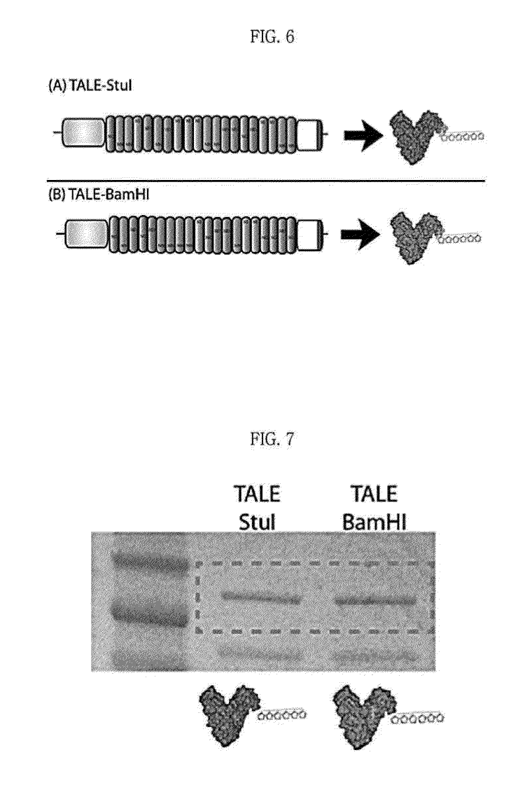

The synthesized TALEN vector was cut to using Sacl (Takara #1078A) and BsaXI (NEB #R0609S) to include the nuclear localization sequence (NLS) and TALE N-terminal and C-terminal sites and blunted using the Quick blunting kit (NEB #E1201S). It was then cut with HindIII (NEB # R0104L), blunted and inserted into AP-treated pET-21 b plasmid (Novagen #69741-3, Novagen, Germany). For purification of the target protein and use as a marker, biotin and a His-tag were inserted at the C-terminal of the TALE domain. The resulting structure is shown in FIG. 6. The tag attached following the TALE C-terminal included the Not I restriction site and the Xho I restriction site and was prepared by Bioneer Co. The biotin (TTA AAT GAT ATA TTT GAG GCA CAA AAA ATT GAA TGG CAT) and the His tag (CAT CAC CAT CAC CAT CAC) were inserted into the pET-21b plasmid after cutting by treating with the Not I (Takara #1166A) and Xho I (Takara #1094A) restriction enzymes to finally obtain a TALE protein expression vector.

### Example 1-3

### Synthesis of TALE Protein (TALE Domain)

A TALE protein was synthesized using the TALE protein expression vector and a bacterial expression system. BL21 E. coli including pET-TALE plasmid was added to 3 mL of a medium containing ampicillin and kept at 37° C. overnight. Then, after transferring to 200 mL of an ampicillin-containing medium and further culturing for 5 hours, 140 μL of 1 M IPTG was added and the culture was kept at 20° C. overnight. The culture medium was transferred to a conical tube and the E. coli cells were collected by centrifugation. The cells were depelleted using 5 mL of a buffer (20 mM Tris-CI, 50 mM NaCl, pH 8.0) and sonicated on ice. After removing E. coli debris through centrifugation, TALE proteins were recovered from the supernatant by binding to Ni-beads (Novagen, Germany), which were then washed and diluted. The TALE protein was concentrated using the 10 kDa MWCO Amicon Ultra column (Millipore, Germany). Finally, a functionalized TALE protein for the target gene was acquired. FIG. 7 shows a result of quantitating the purified StuI-TALE and BamHI-TALE proteins according to the Bradford assay, electrophoresing at 60 V for 3 hours by loading 4 μg of the protein on Any kD™ Mini-PROTEAN® TGX™ precast gel (Bio-Rad, Japan, #456-9033), staining the gel with Bio-Safe Coomassie Stain (Bio-Rad, Japan, #161-0786) for 1 minute and identifying the bands after washing.

### Example 1-4

### Synthesis of TALE Sensor

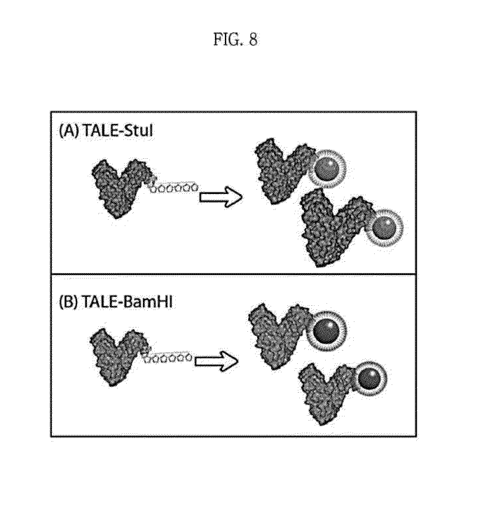

MHPC (1-myristoyl-2-hydroxy-sn-glycero-3-phosphocholine), DPPE-PEG 2000 (1,2-distearoyl-sn-glycero-3-phosphoethanolamine-N-(methoxypolyethylene glycol)-2000) and Ni-NTA (1,2-dioleoyl-snglycero-3-N-(5-amino-1-carboxypentyl) iminodiacetic acid succinyl nickel salt) were added to a solution of quantum dots (QDs) in chloroform at ratios of 80%, 15% and 5%. After adding 2 mL of DI water to the solution at 80° C., the mixture was heated for 1 hour to remove the chloroform. Then, the QDs (surface-treated with MHPC, PEG and Ni-NTA) dispersed in water were sonicated for 1 hour to obtain a suspension of single particles. The prepared QDs and the TALE protein having the His-tag were mixed and incubated for 1 hour. Similarly, streptavidin-bound QDs and the biotin-bound TALE protein were mixed and incubated for 1 hour. FIG. 8 schematically shows the purified TALE domain proteins and the tagging protein sites attached on the QDs.

### Test Example 1

### Detection of Target T7 Bacteriophage Fragments

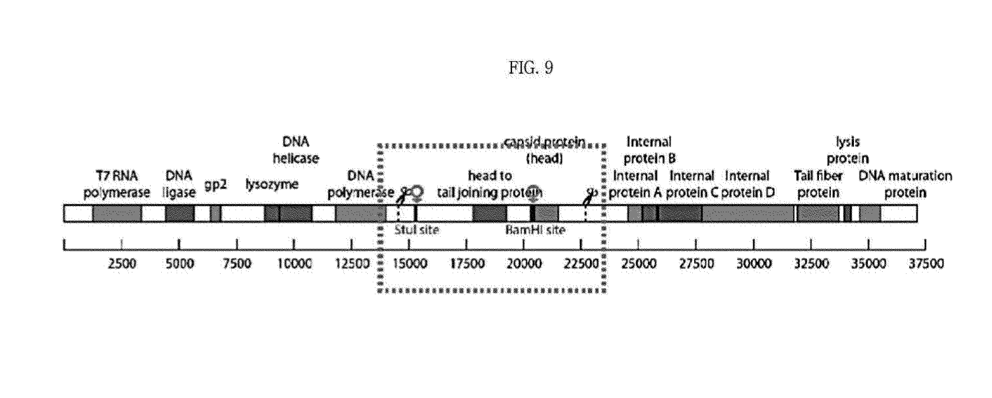

FIG. 9 schematically shows isolation of a site binding to the TALE sensor by cleaving the entire 10-3b T7 bacteriophage sequence.

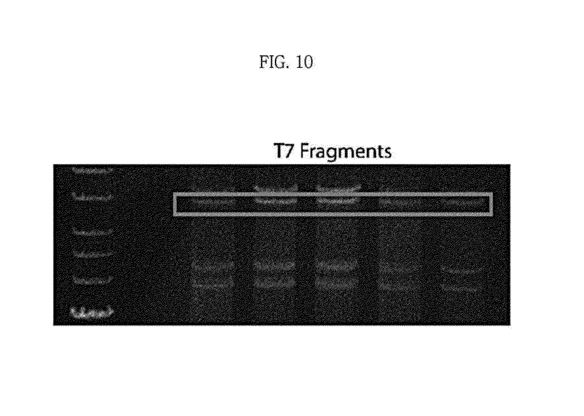

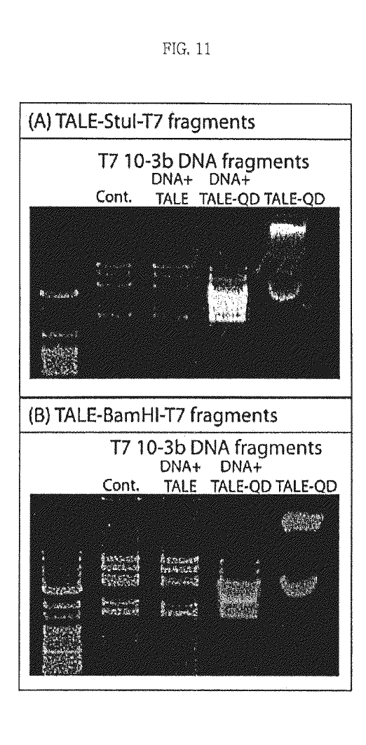

The full sequence of the 10-3b T7 bacteriophage (Novagen #70548-3) was cleaved with the PpuMI (NEB #R0506S) restriction enzyme. The resulting 7730-bp fragment (14978-22707) was cut by the StuI (NEB #R0187S) restriction enzyme which cuts the 15481 site and the BamHI (NEB #R3136S) which cuts the 20410 site. The result is shown in FIG. 10.

As can be seen from FIG. 10, the target sites of the T7 bacteriophage were accurately cleaved.

In addition, the TALE sensor synthesized with the QDs was bound to the T7 bacteriophage fragments and electrophoresed on agarose gel. As can be seen from FIG. 11, fluorescence was observed in the DNA bands of the TALE-QD-DNA samples (BamHI-TALE-QD: green, StuI-TALE-QD: red) as compared to the line of the TALE sensor alone. This result reveals that DNA fragments can be normally detected with the TALE-QD sensor. Since the fluorescence observed in the line of the TALE-QD-DNA bound to the DNA containing the target DNA site matched the band of the target DNA site, it was confirmed that the TALE-QD probe can normally bind to and detect the target DNA.

### Example 2

### Preparation of TALE Domains (dTALE) for Diagnosis of Various Diseases

As shown in Table 2, sensors containing each TALE domain were prepared in the same manner as in Example 1, for the HPV16 E6 gene known to cause cervical cancer, the site where hyper methylation is observed in prostate cancer patients, the H7N9 avian influenza virus, the coxsackie A5 virus VP1 gene causing hand, foot and mouth disease and the HIV gag gene causing AIDS. In Table 2, only the 12th and 13rd amino acids of the 34 amino acids of the TALE repeat unit are shown.

TABLE 2

| TALE domain | Targeting | Amino acid sequence of | | |
|---|---|---|---|---|
| name | sequence | TALE repeat unit | Target gene | Disease name |
| TALEHPV1 | TGATATAATATTA | NGNNNINGNINGNININGNINGN | HPV16 E6-1 | Cervical |
| | GAATGTG | GNINNNININGNNNGNN | | cancer |
| | (SEQ ID NO 5) | (SEQ ID NO 6) | | (HPV16 E6) |
| TALEHPV2 | TCCATATGCTGT | NGHDHDNINGNINGNNHDNGN | HPV16 E6-2 | Cervical |
| | ATGTGATA | NNGNINGNNNGNNNINGNI | | cancer |
| | (SEQ ID NO 7) | (SEQ ID NO 8) | | (HPV16 E6) |
| TALEprostate1 | TGCTGCCGGC | NGNNNGNGNNNGNGNNNNN | ICMT-HES3 | Prostate |
| (methylated C | CTCCCCCCAC | GNGNGNGNGNGNGNGNGNIN | | cancer |
| form) | (SEQ ID NO 9) | G | | |
| | | (SEQ ID NO 10) | | |
| TALEprostate2 | TGGGGACTACA | NGNNNNNNNNNINGNGNINGN | TBR1 | Prostate |
| (methylated C | CCTGTAAAG | INGNGNGNNNGNINININN | | cancer |
| form) | (SEQ ID NO 11) | (SEQ ID NO 12) | | |
| TALEpancreas1 | TCGCCGCCGC | NGHDNNNGNGNNHDNGNNHD | BNC1-1 | Pancreatic |
| (methylated C | CCGCCGCGGA | NGNGNNHDNGNNHDNNNNNI | | cancer |
| form) | (SEQ ID NO 13) | (SEQ ID NO 14) | | |
| TALEpancreas2 | TCCCCGGGAG | NGHDHDHDNGNNNNNNANNA | BNC1-2 | Pancreatic |
| (methylated C | AGGCAAACAC | NNNNHDAAAHDAHD | | cancer |
| form) | (SEQ ID NO 15) | (SEQ ID NO 16) | | |
| TALEcolon1 | TCAGAGGGTCC | NGHDNINNNINNNNNNNGHDH | APC | Colorectal |
| | AGGTTCTTC | DNINNNNNGNGHDNGNGHD | (NM_000038.5) | cancer |
| | (SEQ ID NO 17) | (SEQ ID NO 18) | | |
| TALEcolon2 | TAAAAAGAAAA | NGNINININININNNININININNNI | APC | Colorectal |
| | GATTGGAAC | NGNGNNNNNINIHD | (NM_000038.5) | cancer |
| | (SEQ ID NO 19) | (SEQ ID NO 20) | | |
| TALEH7N9-1 | TGCTTAGTTTG | NGNNHDNGNGNINNNGNGNG | Avian Influenza | Avian influenza |
| | ACTGGGTCA | NNNIHDNGNNNNNNNGHDNI | H7N9 | (H7N9) |
| | (SEQ ID NO 21) | (SEQ ID NO 22) | | |
| TALEH7N9-2 | TGGTTTAGCTT | NGNNNNNGNGNGNINNHDNG | Avian Influenza | Avian influenza |
| | CGGGGCATC | NGHDNNNNNNNNHDNINGHD | H7N9 | (H7N9) |
| | (SEQ ID NO 23) | (SEQ ID NO 24) | | |
| TALEH5N1-1 | TTGGAATGCAG | NGNGNNNNNININGNNHDNINN | Neuraminidase | Avian influenza |
| | AACTTTCTT | NINIHDNGNGNGHDNGNG | (NA) gene | (H5N1) |
| | (SEQ ID NO 25) | (SEQ ID NO 26) | (DQ643810.1) | |
| TALEH5N1-2 | TAAGGATTGGT | NGNININNNNNINGNGNNNNN | Neuraminidase | Avian influenza |
| | TCCAAGGGG | GNGHDHDNININNNNNNNN | (NA) gene | (H5N1) |
| | (SEQ ID NO 27) | (SEQ ID NO 28) | (DQ643810.1) | |
| TALEHFMD1 | TACTGGACCAC | NGNIHDNGNNNNNIHDHDNIHD | Coxsackievirus | Hand, foot and |
| | CTGGCGGCA | HDNGNNNNHDNNNNHDNI | A5 isolate | mouth disease |
| | (SEQ ID NO 29) | (SEQ ID NO 30) | PUMCH5454Ju | (HFMD |
| | | | n07 VP1 gene | CVA5VP1) |
| TALEHFMD2 | TAACCCTCACT | NGNINIHDHDHDNGHDNIHDNG | Coxsackievirus | Hand, foot and |
| | AAAGGGAGA | NINININNNNNNNINNNI | A5 isolate | mouth disease |
| | (SEQ ID NO 31) | (SEQ ID NO 32) | PUMCH5454Ju | (HFMD |
| | | | n07 VP1 gene | CVA5VP1) |
| | | | HIV-1 isolate | |
| TALEHIV-1 | TAGTTAGCCAG | NGNINNNGNGNINNHDHDNINN | P6B_acute_A1 | AIDS |
| | AGAGCTCCC | NINNNINNHDNGHDHDHD | _1A gag | (HIV gag) |
| | (SEQ ID NO 33) | (SEQ ID NO 34) | protein (gag) | |
| | | | gene | |
| | | | HIV-1 isolate | |
| TALEHIV-2 | TAGCTCCCTGC | NGNINNHDNGHDHDHDNGNN | P6B_acute_A1 | AIDS |
| | TTGCCCATA | HDNGNGNNHDHDHDNINGNI | _1A gag | (HIV gag) |
| | (SEQ ID NO 35) | (SEQ ID NO 36) | protein (gag) | |
| | | | gene | |

### Test Example 2

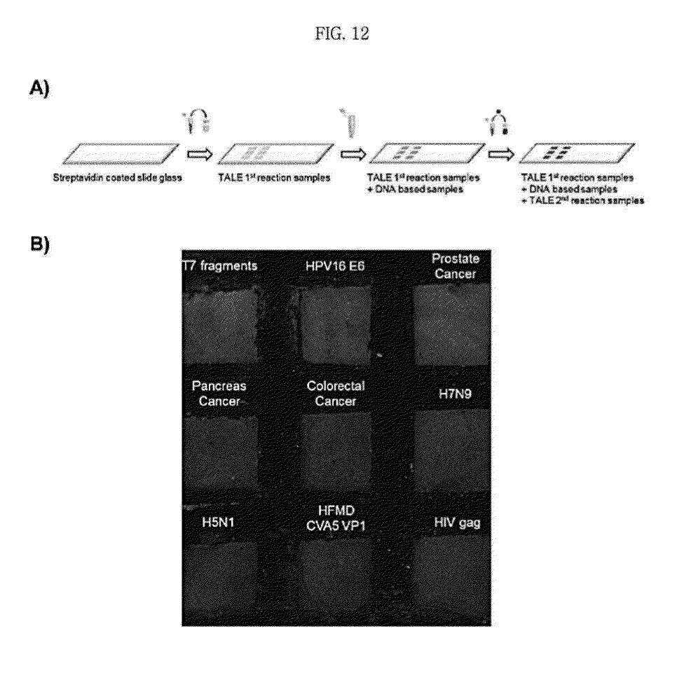

Sandwich targeting type detection test was conducted using the TALE sensors prepared above. For each disease, two or three TALE sensors were selected and one of the TALE sensors containing biotin was bound onto a streptavidin-coated slide glass. After washing off the unbound TALE sensor, solution samples containing target sites were dropped onto the slide glass. After a predetermined time, the unbound samples were washed off. Then, the other TALE sensors except the one used for coating on the slide glass were dropped onto the slide glass and reacted for a predetermined time. The TALE sensors are those attached to QDs that can emit fluorescence signals at the biotin or HIS tag site. After washing off the unbound TALE sensors, fluorescence was observed (FIG. 12-A). As can be seen from FIG. 12-B, fluorescence was observed from the samples having target sites and it was confirmed that the TALE sensor prepared according to the present disclosure can detect the target site.

In FIG. 12-B, the sites where green fluorescence is observed are those where binding to the target DNA occurred. Accordingly, it can be seen that specific diseases can be easily detected or diagnosed according to the present disclosure.

What is claimed is:

1. A kit for detection or diagnosis of a disease, comprising a dTALE (designed transcription activator-like effector) domain which binds to a disease-causing gene.

2. The kit according to claim 1, wherein the dTALE domain comprises 2-30 TALE repeat units.

3. The kit according to claim 1, wherein the composition further comprises a tag, the dTALE domain and the tag are present in the composition in the form of a complex, and the tag and the dTALE domain bind at a site different from the binding site of the dTALE domain with the gene.

4. The kit according to claim 3, wherein the tag is an HIS tag, a CYS tag, a GST tag or a biotin binding tag.

5. The kit according to claim 3, wherein the composition further comprises a detectable labeling agent and the detectable labeling agent binds to the tag in the complex.

6. The kit according to claim 5, wherein the detectable labeling agent is a quantum dot, a magnetic bead nanoparticle, a gold nanoparticle, a fluorescent dye, a fluorescent protein, a nanophosphor or a silicon nanoparticle.

7. The kit according to claim 1, wherein the disease is cancer, avian influenza, hand, foot and mouth disease or AIDS.

8. The kit according to claim 7, wherein the cancer is pancreatic cancer, colorectal cancer, cervical cancer or prostate cancer.

9. The kit according to claim 1, wherein the gene comprises one or more DNA strand.

10. The kit according to claim 9, wherein the gene is one or more selected from a group consisting of a single-stranded DNA, a double-stranded DNA, a circular DNA, a free DNA and a DNA-RNA hybrid.

11. A method for detection or diagnosis of a disease, comprising treating a sample obtained from an individual with the composition according to claim 5.

12. The method for detection or diagnosis of a disease according to claim 11, wherein the sample is blood or urine.

13. The method for detection or diagnosis of a disease according to claim 11, wherein the gene comprises one or more DNA strand.

14. The method for detection or diagnosis of a disease according to claim 13, wherein the gene is one or more selected from a group consisting of a single-stranded DNA, a double-stranded DNA, a circular DNA, a free DNA and a DNA-RNA hybrid.

15. The method for detection or diagnosis of a disease according to claim 11, wherein the disease is cancer, avian influenza, hand, foot and mouth disease or AIDS.

16. The method for detection or diagnosis of a disease according to claim 15, wherein the cancer is pancreatic cancer, colorectal cancer, cervical cancer or prostate cancer.


SEQUENCE LISTING (36 sequences)

SEQ ID NO: 1 - protein, 34 aa, Artificial Sequence
  TALE repeated unit for binding with Nucleotide A
LTPEQVVAIASNIGGKQALETVQRLLPVLCQAHG

SEQ ID NO: 2 - protein, 34 aa, Artificial Sequence
  TALE repeated unit for binding with Nucleotide T or mC
LTPEQVVAIASNGGGKQALETVQRLLPVLCQAHG

SEQ ID NO: 3 - protein, 34 aa, Artificial Sequence
  TALE repeated unit for binding with Nucleotide G
LTPEQVVAIASNNGGKQALETVQRLLPVLCQAHG

SEQ ID NO: 4 - protein, 34 aa, Artificial Sequence
  TALE repeated unit for binding with Nucleotide C
LTPEQVVAIASHDGGKQALETVQRLLPVLCQAHG

SEQ ID NO: 5 - DNA, 20 bp, Human Papillomavirus type 16
tgatataatattagaatgtg

SEQ ID NO: 6 - protein, 40 aa, Artificial Sequence
  TALE repeated unit for binding with sequence number 5
NGNNNINGNINGNININGNINGNGNINNNININGNNNGNN

SEQ ID NO: 7 - DNA, 20 bp, Human Papillomavirus type 16
tccatatgctgtatgtgata

SEQ ID NO: 8 - protein, 40 aa, Artificial Sequence
  TALE repeated unit for binding with sequence number 7
NGHDHDNINGNINGNNHDNGNNNGNINGNNNGNNNINGNI

SEQ ID NO: 9 - DNA, 20 bp, Homo Sapiens
tgctgccggcctccccccac

SEQ ID NO: 10 - protein, 40 aa, Artificial Sequence
  TALE repeated unit for binding with sequence number 9
NGNNNGNGNNNGNGNNNNNGNGNGNGNGNGNGNGNGNING

SEQ ID NO: 11 - DNA, 20 bp, Homo Sapiens
tggggactacacctgtaaag

SEQ ID NO: 12 - protein, 40 aa, Artificial Sequence
  TALE repeated unit for binding with sequence number 11
NGNNNNNNNNNINGNGNINGNINGNGNGNNNGNINININN

SEQ ID NO: 13 - DNA, 20 bp, Homo Sapiens
tcgccgccgcccgccgcgga

SEQ ID NO: 14 - protein, 40 aa, Artificial Sequence
  TALE repeated unit for binding with sequence number 13
NGHDNNNGNGNNHDNGNNHDNGNGNNHDNGNNHDNNNNNI

SEQ ID NO: 15 - DNA, 20 bp, Homo Sapiens
tccccgggagaggcaaacac

SEQ ID NO: 16 - protein, 40 aa, Artificial Sequence
  TALE repeated unit for binding with sequence number 15
NGHDHDHDNGNNNNNNNINNNINNNNHDNININIHDNIHD

SEQ ID NO: 17 - DNA, 20 bp, Homo Sapiens
tcagagggtccaggttcttc

SEQ ID NO: 18 - protein, 40 aa, Artificial Sequence
  TALE repeated unit for binding with sequence number 17
NGHDNINNNINNNNNNNGHDHDNINNNNNGNGHDNGNGHD

SEQ ID NO: 19 - DNA, 20 bp, Homo Sapiens
taaaaagaaaagattggaac

SEQ ID NO: 20 - protein, 40 aa, Artificial Sequence
  TALE repeated unit for binding with sequence number 19
NGNINININININNNININININNNINGNGNNNNNINIHD

SEQ ID NO: 21 - DNA, 20 bp, Avian Influenza H7N9
tgcttagtttgactgggtca

SEQ ID NO: 22 - protein, 40 aa, Artificial Sequence
  TALE repeated unit for binding with sequence number 21
NGNNHDNGNGNINNNGNGNGNNNIHDNGNNNNNNNGHDNI

SEQ ID NO: 23 - DNA, 20 bp, Avian Influenza H7N9
tggtttagcttcggggcatc

SEQ ID NO: 24 - protein, 40 aa, Artificial Sequence
  TALE repeated unit for binding with sequence number 23
NGNNNNNGNGNGNINNHDNGNGHDNNNNNNNNHDNINGHD

SEQ ID NO: 25 - DNA, 20 bp, Avian Influenza H5N1
ttggaatgcagaactttctt

SEQ ID NO: 26 - protein, 40 aa, Artificial Sequence
  TALE repeated unit for binding with sequence number 25
NGNGNNNNNININGNNHDNINNNINIHDNGNGNGHDNGNG

SEQ ID NO: 27 - DNA, 20 bp, Avian Influenza H5N1
taaggattggttccaagggg

SEQ ID NO: 28 - protein, 40 aa, Artificial Sequence
  TALE repeated unit for binding with sequence number 27
NGNININNNNNINGNGNNNNNGNGHDHDNININNNNNNNN

SEQ ID NO: 29 - DNA, 20 bp, Coxsackievirus A5
tactggaccacctggcggca

SEQ ID NO: 30 - protein, 40 aa, Artificial Sequence
  TALE repeated unit for binding with sequence number 29
NGNIHDNGNNNNNIHDHDNIHDHDNGNNNNHDNNNNHDNI

SEQ ID NO: 31 - DNA, 20 bp, Coxsackievirus A5
taaccctcactaaagggaga

SEQ ID NO: 32 - protein, 40 aa, Artificial Sequence
  TALE repeated unit for binding with sequence number 31
NGNINIHDHDHDNGHDNIHDNGNINININNNNNNNINNNI

SEQ ID NO: 33 - DNA, 20 bp, Human Immunodeficiency Virus type 1
tagttagccagagagctccc

SEQ ID NO: 34 - protein, 40 aa, Artificial Sequence
  TALE repeated unit for binding with sequence number 33
NGNINNNGNGNINNHDHDNINNNINNNINNHDNGHDHDHD

SEQ ID NO: 35 - DNA, 20 bp, Human Immunodeficiency Virus type 1
tagctccctgcttgcccata

SEQ ID NO: 36 - protein, 40 aa, Artificial Sequence
  TALE repeated unit for binding with sequence number 35
NGNINNHDNGHDHDHDNGNNHDNGNGNNHDHDHDNINGNI